(19) United States
(12) Patent Application Publication
(10) Pub. No.: US 2025/0161249 A1
(43) Pub. Date: May 22, 2025

(54) USE OF PHENYLLACTIC ACID IN INHIBITING HELICOBACTER PYLORI INFECTION

(71) Applicant: Zhejiang Gongshang University, Hangzhou (CN)

(72) Inventors: Qing Gu, Hangzhou (CN); Wanxin Cui, Hangzhou (CN); Qingqing Zhou, Hangzhou (CN); Ping Li, Hangzhou (CN); Danli Wu, Hangzhou (CN); Chenlan Xia, Hangzhou (CN); Ziqi Chen, Hangzhou (CN); Ying Liang, Hangzhou (CN)

(21) Appl. No.: 19/027,214

(22) Filed: Jan. 17, 2025

Related U.S. Application Data
(63) Continuation of application No. PCT/CN2023/090828, filed on Apr. 26, 2023.

(30) Foreign Application Priority Data
Sep. 11, 2022 (CN) 202211106356.4

(51) Int. Cl. A61K 31/192; A61P 1/04; A61P 31/04

(52) U.S. Cl. CPC A61K 31/192; A61P 1/04; A61P 31/04

(57) ABSTRACT

The present invention belongs to the technical field of microorganisms. Provided in the present invention is use of phenyllactic acid in preparation of a drug for inhibiting Helicobacter pylori infection. The phenyllactic acid inhibits antibiotic-resistant Helicobacter pylori infection, and particularly can inhibit growth of Helicobacter pylori insensitive to metronidazole.

### CROSS-REFERENCE TO RELATED APPLICATIONS

The present application is a continuation application of PCT application No. PCT/CN2023/090828 filed on Apr. 26, 2023, which claims the benefit of Chinese Patent Application No. 202211106356.4 filed on Sep. 11, 2022. The contents of all of the aforementioned applications are incorporated by reference herein in their entirety.

### TECHNICAL FIELD

The present invention relates to use of phenyllactic acid in inhibiting gastric mucosal inflammation caused by Helicobacter pylori infection and improving gastric microecology, belonging to the technical field of microorganisms.

### BACKGROUND

Helicobacter pylori (H. pylori), a gram-negative bacterium that can colonize epithelial cells of human gastric mucosa, can cause diseases such as gastritis and peptic ulcer after infection, and is also a potential factor inducing gastric cancer, which is classified as a class I carcinogen by the International Agency for Research on Cancer (IARC).

With the increasing rate of H. pylori infection, the chance of secondary infection also increases year by year. At present, the most standard and most commonly used method for treating H. pylori infection is the “triple” therapy of clarithromycin and amoxicillin in combination with a proton pump inhibitor (PPI). Recent data statistics have shown that the drug resistance rate of a part of H. pylori to clarithromycin and metronidazole is as high as 94.1% and 67.6%, and the eradication rate of H. pylori by standard triple therapy is less than 80%. In addition, patients often have serious adverse reactions (such as abdominal pain, nausea, diarrhea and the like) due to the wide use of antibiotics. It is therefore particularly important to find a safe and side-effect-free solution to alleviate H. pylori infection.

Phenyllactic acid (PLA) is a novel natural small molecular organic acid produced by the metabolism of lactic acid bacteria, and is widely favored in the food field, pharmaceutical field and cosmetics industry due to its advantages of stability, safety, good solubility and the like. However, whether phenyllactic acid has an inhibitory effect on H. pylori has not been reported.

### SUMMARY

A technical problem to be solved by the present invention is to provide phenyllactic acid (PLA) for inhibiting antibiotic-resistant Helicobacter pylori infection.

In order to solve the above technical problem, the present invention provides use of phenyllactic acid in preparation of a drug for inhibiting Helicobacter pylori (H. pylori) infection.

As an improvement of the use of the present invention, the phenyllactic acid inhibits antibiotic-resistant H. pylori infection.

As a further improvement of the use of the present invention, the phenyllactic acid is capable of inhibiting growth of H. pylori insensitive to metronidazole (resistant to metronidazole).

As a further improvement of the use of the present invention, the phenyllactic acid has at least one of the following properties:
the phenyllactic acid is capable of inhibiting the growth of H. pylori;
the phenyllactic acid relieves gastric mucosal inflammation caused by the H. pylori infection;
the phenyllactic acid inhibits urease activity of the H. pylori; and
the phenyllactic acid is destructive to a bacterial cell of the H. pylori.

The H. pylori is, for example, Helicobacter pylori ZJC03.

At present, the in vitro inhibitory effect of phenyllactic acid on H. pylori, and the regulatory effect of phenyllactic acid on gastric inflammation caused by H. pylori infection and gastric flora are not reported in the prior art.

However, the present invention proves that the phenyllactic acid has a significant inhibitory property against the H. pylori; the minimum inhibitory concentration (MIC) range of the phenyllactic acid for the H. pylori is 2.5 mg/mL; the phenyllactic acid inhibits the urease activity of the H. pylori; and the phenyllactic acid is destructive to the bacterial cell of the H. pylori.

The use of the present invention includes that the phenyllactic acid repairs gastric mucosal injury caused by the H. pylori infection, alleviates the gastric mucosal inflammation caused by the H. pylori infection and improves gastric dysbacteriosis. The gastric mucosal injury is observed by H&E pathological staining and evaluated through tissue injury in the gastric mucosa; and a pathological degree of the gastric mucosal inflammation is related to expression levels of inflammatory factors, including inflammatory factors TNF-α, IFN-γ, IL-6 and IL-10.

The effects of the phenyllactic acid on the gastric microecology in the present invention include decreasing the increase of Proteobacteria caused by the H. pylori infection, decreasing the proportion of Helicobacter, and significantly improving the contents of Lactobacillus, Bifidobacterium and Prevotella in the stomach.

The phenyllactic acid is orally administrated with a recommended dose of 2.5-3.5 mg/person per day.

Compared with the prior art, the present invention has the following advantages:

1. in vitro experiments of the present invention prove that the phenyllactic acid has an inhibitory effect on the growth of the H. pylori, and it is speculated according to the experiments that the bacteriostatic effect of the phenyllactic acid is exerted by destroying the wall membranes of the bacteria and inhibiting the urease activity of the H. pylori.

2. in mouse experiments, the phenyllactic acid can relieve the gastric mucosal injury caused by the H. pylori infection, down-regulate the expression levels of proinflammatory factors IL-1B, IL-6 and IFN-γ, and up-regulate the expression levels of an anti-inflammatory factor IL-10.

3. the therapeutic effect of the phenyllactic acid improves the abundances of probiotics in the gastric mucosa of mice and reduces the abundances of pathogenic bacteria, having a good effect on the recovery of gastric microecological flora after the H. pylori infection.

In conclusion, the present invention identifies new use of the phenyllactic acid in inhibiting gastritis caused by the H. pylori infection through in vitro and in vivo experimental studies. Therefore, the phenyllactic acid has the potential for application in drug development.

### BRIEF DESCRIPTION OF THE DRAWINGS

The specific implementations of the present invention will be further described in detail below with reference to the accompanying drawings.

FIG. 1 is a schematic diagram showing effects of sub-inhibitory concentrations of phenyllactic acid on growth curves of H. pylori;

FIG. 2 is a schematic diagram showing inhibition rates of urease activity of H. pylori by different concentrations of phenyllactic acid;

FIG. 3 shows scanning electron microscope images before and after treatment of H. pylori with phenyllactic acid,
where in FIG. 3:
A is a scanning electron microscope image of untreated H. pylori (×15,000);
B is a scanning electron microscope image of untreated H. pylori (×30,000);
C is a scanning electron microscope image of H. pylori after treatment with phenyllactic acid at a minimum inhibitory concentration (MIC) (×15,000); and
D is a scanning electron microscope image of H. pylori after treatment with phenyllactic acid at an MIC (×30,000);

FIG. 4 shows transmission electron microscope images before and after treatment of H. pylori with phenyllactic acid,
where in FIG. 4:
A is a transmission electron microscope image of untreated H. pylori (×15,000);
B is a transmission electron microscope image of untreated H. pylori (×30,000);
C is a transmission electron microscope image of H. pylori after treatment with phenyllactic acid at an MIC (×15,000); and
D is a transmission electron microscope image of H. pylori after treatment with phenyllactic acid at an MIC (×30,000);

FIG. 5 shows negative-stain transmission electron microscope images before and after treatment of H. pylori with phenyllactic acid,
where in FIG. 5:
A is a negative-stain transmission electron microscope image of untreated H. pylori (×20,000);
B is a negative-stain transmission electron microscope image of untreated H. pylori (×20,000);
C is a negative-stain transmission electron microscope image of H. pylori after treatment with phenyllactic acid at an MIC (×20,000); and
D is a negative-stain transmission electron microscope image of H. pylori after treatment with phenyllactic acid at an MIC (×20,000);

FIG. 6 is a schematic diagram showing treatment time of mice in an animal experiment;

FIG. 7 shows schematic diagrams of HE pathological staining of gastric mucosal tissues of each group of mice,
where in FIG. 7:
A is an HE pathological staining image of gastric mucosal tissues of mice in an untreated normal control group;
B is an HE pathological staining image of gastric mucosal tissues of mice in an untreated normal control group;
C is an HE pathological staining image of gastric mucosal tissues of mice in an H. pylori infection group;
D is an HE pathological staining image of gastric mucosal tissues of mice in an H. pylori infection group;
E is an HE pathological staining image of gastric mucosal tissues of mice in a phenyllactic acid treatment group;
F is an HE pathological staining image of gastric mucosal tissues of mice in a phenyllactic acid treatment group;
G is an HE pathological staining image of gastric mucosal tissues of mice in an antibiotic treatment group; and
H is an HE pathological staining image of gastric mucosal tissues of mice in an antibiotic treatment group;

FIG. 8 shows schematic diagrams of RT-qPCR detection of gastric mucosal tissues of mice, where in FIG. 8:
A is a schematic diagram of RT-qPCR detection of IL-1ß in the gastric mucosal tissues of the mice;
B is a schematic diagram of RT-qPCR detection of IFN-γ in the gastric mucosal tissues of the mice;
C is a schematic diagram of RT-qPCR detection of IL-6 in the gastric mucosal tissues of the mice; and
D is a schematic diagram of RT-qPCR detection of IL-10 in the gastric mucosal tissues of the mice;

FIG. 9 shows a schematic diagram of distribution analysis of mouse gastric flora at “phylum” and “genus” levels;
where in FIG. 9:
A is a schematic diagram of distribution analysis of the mouse gastric flora at the “phylum” level; and
B is a schematic diagram of distribution analysis of the mouse gastric flora at the “genus” level;

FIG. 10 is a schematic diagram showing Beta diversity analysis of mouse gastric flora; and

FIG. 11 shows schematic diagrams of a clustering tree diagram and LDA score analysis for significant difference analysis of gastric flora species of each group of mice;
where in FIG. 11:
A is a schematic diagram of the clustering tree diagram for the significant difference analysis of the gastric flora species of each group of mice; and
B is a schematic diagram of the LDA score analysis for the significant difference analysis of the gastric flora species.

### DETAILED DESCRIPTION

The present invention is further described below in conjunction with specific examples, and the advantages and characteristics of the present invention will become clearer with the description. However, these examples are only exemplary and do not set any limitation to the scope of the present invention.

The conventional culture mediums and reagents involved in the following examples are as follows:

Colombia blood agar base (CBA) medium: 5.2 g of Columbia medium (Qingdao Hopebio) was weighed in 100 mL of ultrapure water, subjected to autoclaved sterilization at 121° C. for 15 minutes, and cooled to 44-55° C. 7 mL of sterile sheep blood (Shanghai Yuanye Biotechnology Co., Ltd.) and 1 mL of a Helicobacter pylori (H. pylori) additive (Qingdao Hopebio) were added. After being coagulated, the medium was prepared for late use.

modified Brucella broth medium: 28.6 g of Brucella broth medium (Qingdao Hopebio) was weighed, heated and dissolved in 1 L of ultrapure water, subjected to autoclaved sterilization at 121° C. for 15 minutes, and prepared for later use.

urease medium: 0.1 g of yeast powder, 9.1 g of monopotassium phosphate, 9.5 g of disodium hydrogen phosphate, 20.0 g of urea and 0.01 g of phenol red were mixed, the pH value was adjusted to 6.5, and the mixture was set to a volume by ultrapure water into a 1-L volumetric flask.

formulation of antibiotics for triple therapy: antibiotics were converted to the doses used in mice according to the instructions and formulated. 2.1 g of clarithromycin, 82.5 mg of omeprazole and 12.5 g of amoxicillin were dissolved in 1 L of ultrapure water, fully and uniformly mixed, and stored in an environment at 4° C. for later use.

A method for preparing bacterial cells of H. pylori involved in the following examples is as follows:
after frozen H. pylori ZJC03 was thawed at room temperature, the H. pylori was coated and inoculated on the prepared CBA medium and then placed into a culture box (containing a microaerophilic gas-generating pack, Mitsubishi Gas Chemical Company, Inc.) to be cultured for 48-72 h at 37° C. for resuscitation. After two generations of activation, the bacterial cells were gently washed down by the modified Brucella broth to prepare 10⁸ CFU/mL of a bacterial suspension.

The H. pylori is Helicobacter pylori ZJC03, and the deposit information thereof is as follows: deposit name: Helicobacter pylori ZJC03, deposit unit: China Center for Type Culture Collection, deposit address: Wuhan University, Wuhan, China, deposit number: CCTCC NO: M 20211218, deposit date: Sep. 26, 2021. This strain is also disclosed in patent CN 112080444 A entitled “LACTOBACILLUS FOR PREVENTING AND TREATING GASTRITIS CAUSED BY HELICOBACTER PYLORI AND USE THEREOF”.

### Example 1 Antimicrobial Susceptibility Test of H. pylori ZJC03

The sensitivity of H. pylori ZJC03 was tested by using an E-text test paper. The results showed that the clinical strain H. pylori ZJC03 had sensitivities to clarithromycin, amoxicillin and tetracycline of 0.8 μg/mL, 0.016 μg/mL and 0.023 μg/mL respectively, and was insensitive to metronidazole.

TABLE 1
Sensitivity of H. pylori ZJC03 to antibiotics

| Antibiotic | Sensitivity |
|---|---|
| Clarithromycin | 0.8 μg/mL |
| Amoxicillin | 0.016 μg/mL |
| Tetracycline | 0.023 μg/mL |
| Metronidazole | / |

### Example 2 Bacteriostasis Experiment by Kirby-Bauer Method

Preparation of different concentrations of phenyllactic acid: phenyllactic acid was diluted two-fold to 40 mg/mL, 20 mg/mL, 10 mg/mL, 5 mg/mL, 2.5 mg/mL and 1.25 mg/mL. The solvent used for dilution was ultrapure water.

Preparation of antimicrobial susceptibility disks: filter paper was made into circular disks with a diameter of 6 mm, subjected to autoclaved sterilization for 15 min and dried. Each circular filter paper disk was completely soaked in phenyllactic acid solutions with different concentrations, and ultrapure water was taken as a blank control to obtain antimicrobial susceptibility disks with different concentrations.

Coating and culture: 100 μL of 10⁸ CFU/mL of an H. pylori bacterial solution was evenly coated on a CBA medium and dried. Then the antimicrobial susceptibility disks with different concentrations of the phenyllactic acid were pasted on a plate by tweezers, and this operation was repeated 3 times for disks of each concentration. Then the plate was placed in a culture box (containing a microaerophilic gas-generating pack inside) and statically cultured at 37° C. for 72 h. The size of an inhibition zone (mm) was measured and the measurement result was recorded.

The bacteriostatic effect of the phenyllactic acid on the strain H. pylori ZJC03 was evaluated by measuring the diameter of the inhibition zone, where the 40 mg/mL phenyllactic acid concentration group had a relatively strong bacteriostatic effect on the H. pylori, with the diameter of the inhibition zone >15 mm; the 5 mg/mL concentration group and the 2.5 mg/mL concentration group had the diameters of the inhibition zones >6 mm; and the 1.25 mg/mL concentration group and the solvent control group had no inhibition zone.

TABLE 2
Effects of different concentrations of
phenyllactic acid on growth of H. pylori

| | Inhibition zone |
|---|---|
| Group | diameter (mm) |
| Solvent control group | / |
| 40 mg/mL of phenyllactic acid solution | 15.35 ± 0.94 |
| 20 mg/mL of phenyllactic acid solution | 12.41 ± 0.14 |
| 10 mg/mL of phenyllactic acid solution | 9.98 ± 0.97 |
| 5 mg/mL of phenyllactic acid solution | 7.51 ± 0.74 |
| 2.5 mg/mL of phenyllactic acid solution | 6.94 ± 0.95 |
| 1.25 mg/mL of phenyllactic acid solution | / |

Note: the diameter of filter paper disks is 6 mm.

### Example 3 Determination of Minimum Inhibitory Concentration (MIC)

Agar dilution method adopted: a sterilized Colombia blood agar medium was uniformly mixed with phenyllactic acid solutions with different concentration gradients (so that the final concentrations of phenyllactic acid were respectively 40 mg/mL, 20 mg/mL, 10 mg/mL, 5 mg/mL, 2.5 mg/mL and 1.25 mg/mL), 100 μL of 10⁸ CFU·mL⁻¹ of an H. pylori bacterial suspension was coated, the mixture was placed in a microaerobic environment for culture at 37° C. for 48 h, the growth condition of H. pylori was observed, and the lowest concentration of the phenyllactic acid at which no bacteria grew in the medium was taken as an MIC value.

The determination results showed that when the concentration of the phenyllactic acid in the medium was higher than 2.5 mg/mL, the surface of the CBA medium was smooth and no H. pylori cells appeared, and when the concentration of the phenyllactic acid in the medium was lower than 2.5 mg/mL, H. pylori bacterial cells appeared on the CBA medium, which was visible to the naked eyes.

### Example 4 Effects of Sub-Inhibitory Concentrations of Phenyllactic Acid on Growth Curves of H. pylori

According to the determination of the bacteriostatic ability, the effects of phenyllactic acid on growth curves were analyzed. An H. pylori suspension cultured overnight was inoculated into a sterilized modified Brucella broth medium. The concentration was adjusted such that the initial inoculum size of the bacteria was 1×10⁸ CFU/mL. The phenyllactic acid was added at the final concentrations of ⅛ MIC, ¼ MIC, ½ MIC and MIC, and no drug was added to the control group. The mixed solution was placed in a microaerobic environment at 37° C. for 120 h, the OD₅₆₀ value was determined every other 12 h, and the changes of the growth curves at sub-inhibitory concentrations were observed and recorded.

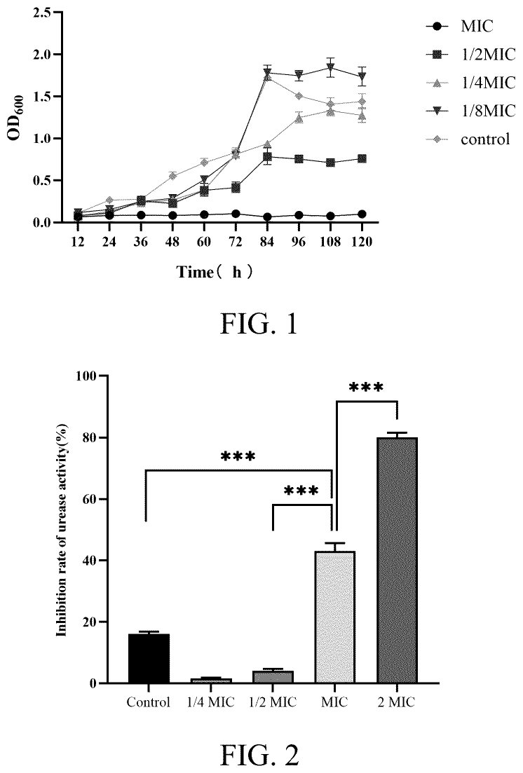

The experimental results are shown in FIG. 1. With the increase of the growth time of H. pylori, the absorbances of the H. pylori increased to different degrees under the action of the phenyllactic acid with different sub-inhibitory concentrations. With the increase of time, the absorbance values all decreased with the increasing concentration of the phenyllactic acid, which meant that the number of bacteria was getting smaller and smaller. Besides, compared with the control group, the growth of the H. pylori was inhibited to different degrees after the action of the phenyllactic acid with different sub-inhibitory concentrations. Within different time, the absorbance values all decreased with increasing drug concentrations, which meant that the number of the bacteria decreased. This difference was not significant at 12 h and 24 h. However, at 48 h, 72 h, 96 h and 120 h and the concentrations of the phenyllactic acid of ½ MIC and MIC, the phenyllactic acid showed a strong bactericidal effect and the growth of the H. pylori was significantly inhibited. When the concentration was reduced to ¼ MIC, the phenyllactic acid showed a certain bactericidal effect, and when the concentration was at ⅛ MIC, the growth of the H. pylori was basically not inhibited.

### Example 5 Determination of Urease Activity of H. pylori Inhibited by Phenyllactic Acid

The urease activity of H. pylori inhibited by phenyllactic acid was determined by using a phenol red method. The H. pylori was activated and cultured, and washed 2 times with a Brucella broth medium. The concentration of the H. pylori was regulated to be 1×10⁸ CFU/mL. A total of 50 μL of the H. pylori culture solution and 50 μL of the phenyllactic acid at the concentrations of ¼ MIC, ½ MIC, MIC and 2 MIC were sequentially added to a 96-well microtiter plate. Then after 100 μL of the mixture was added into 100 μL of a urease medium and uniformly mixed, the mixture was co-cultured for 48 h to observe the color change, the absorbance at 560 nm was determined by a spectrophotometer. In the blank group, only the urease medium was used and the result was determined. In the control group, ultrapure water was used to replace the phenyllactic acid solution and the result was determined. The inhibition rate was calculated by the following formula:

$$Inhibition rate ( \% ) = \frac{( {A}_{u} Determination group ) - ( {A}_{u} Control group )}{{A}_{u} Determination group} \times 100$$

where Au is the urease activity of H. pylori ZJC03.

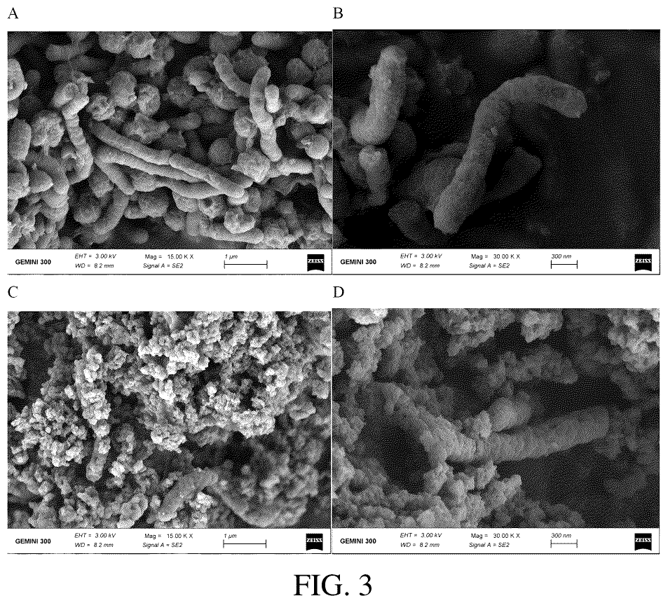

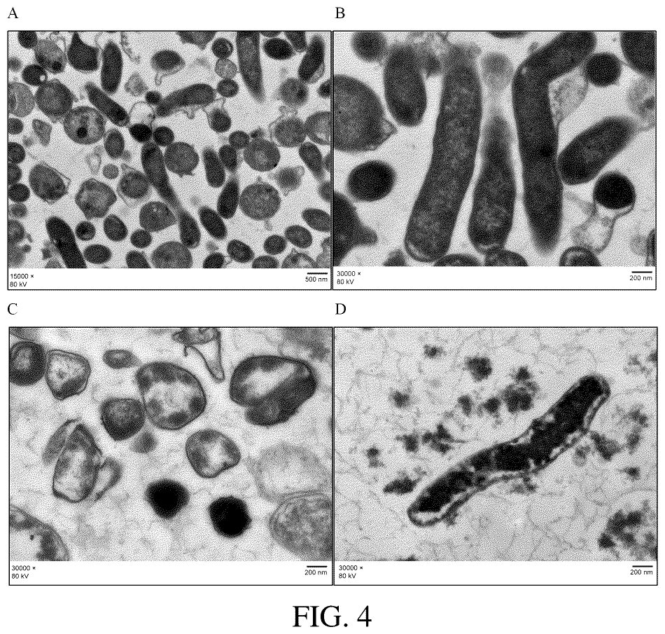

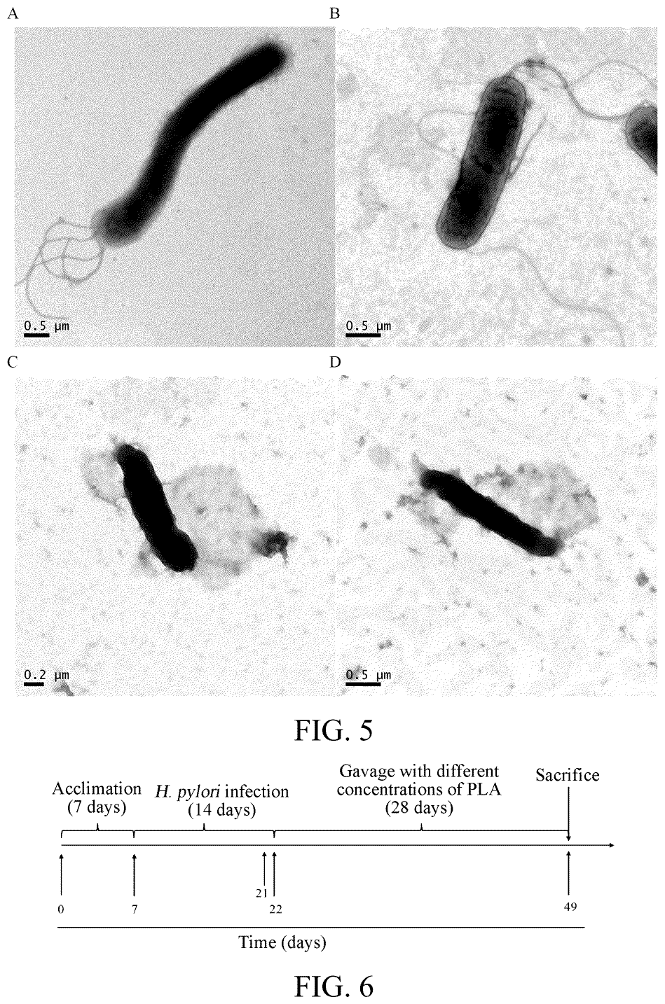

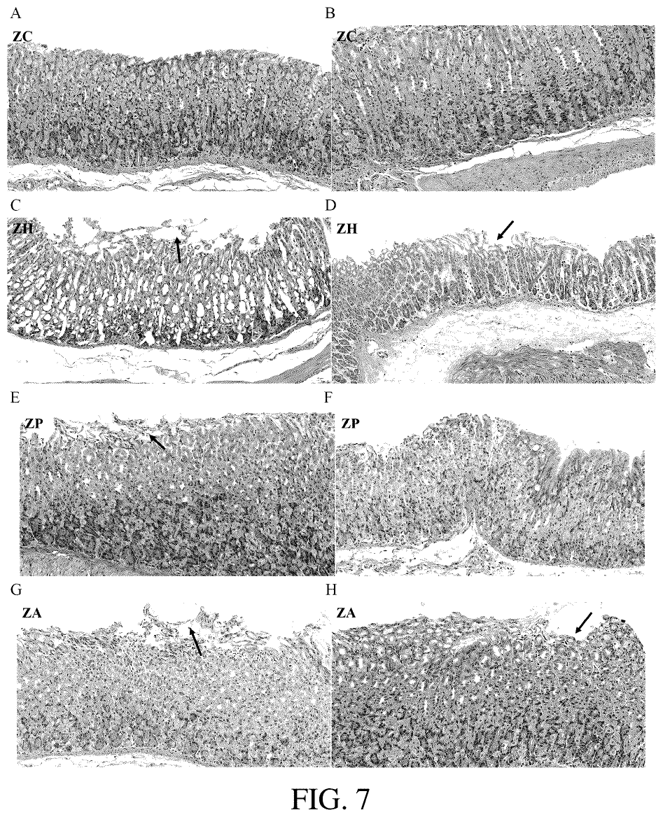

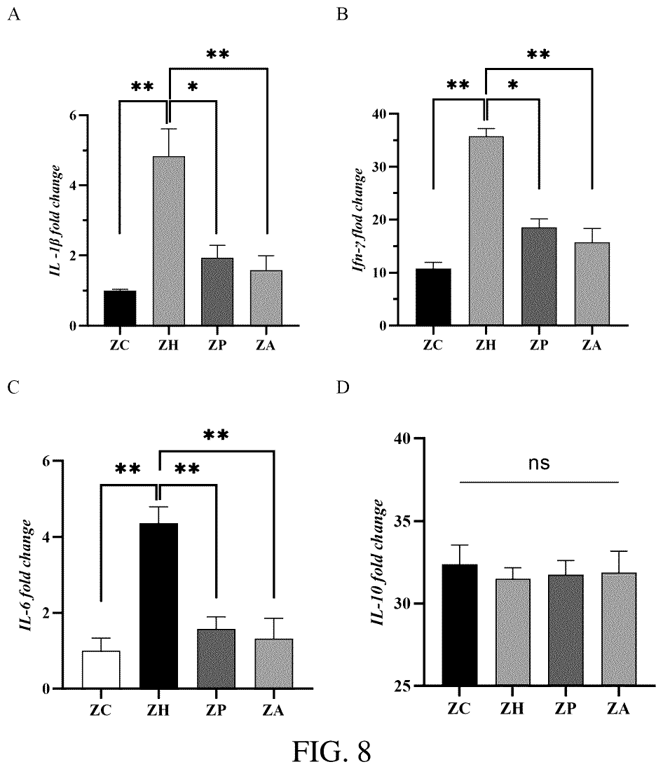

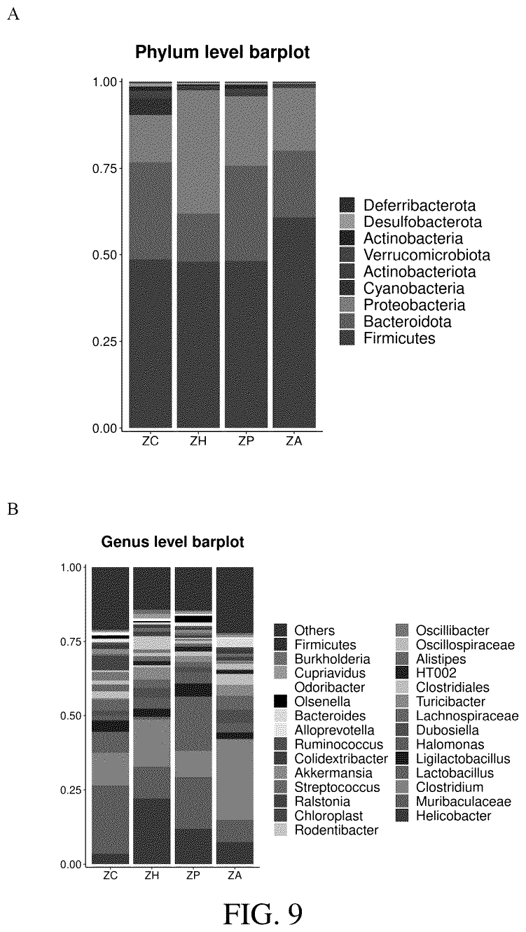

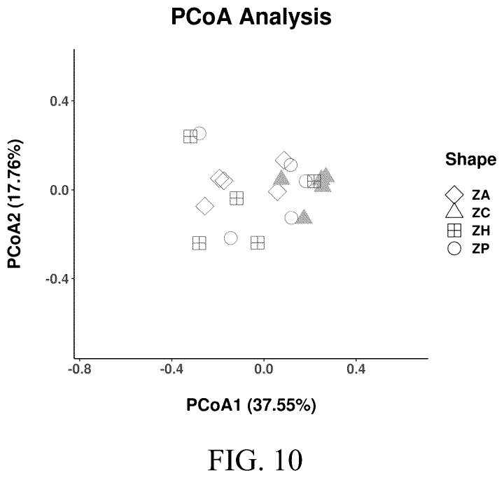

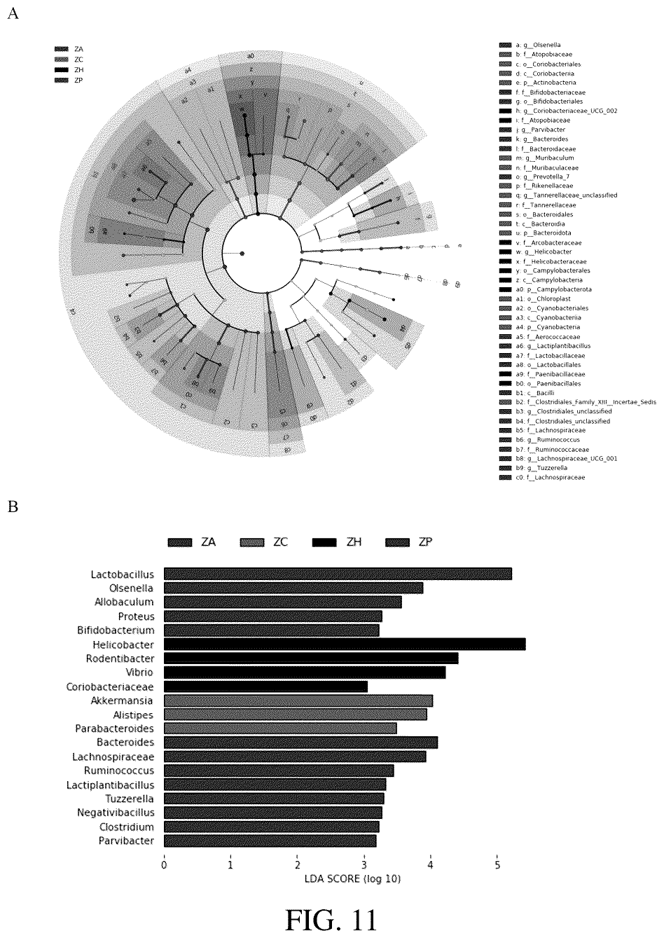

Studies show that when the H. pylori enters the gastric mucosal layer and is subjected to an acid shock (pH<3), the survival of the H. pylori depends on the urease activity of the H. pylori. The enzyme can convert urea in the body into ammonia gas and bicarbonate to neutralize gastric acid, thereby promoting the growth and colonization of the H. pylori. In addition, the urease produced by the H. pylori is also involved in the nitrogen metabolism process, affecting the growth process of host cells, including cell lysis. The studies of the present invention showed that the phenyllactic acid can inhibit the growth of the H. pylori by inhibiting the urease activity of the H. pylori. As shown in FIG. 2, the urease activity of the H. pylori decreased with increasing concentration under the treatment of the phenyllactic acid at different concentrations. After the phenyllactic acid and the H. pylori were co-cultured for 48 h, the inhibition rate of the phenyllactic acid at the MIC was 43.01%±2.11%, the inhibition rate of the phenyllactic acid at 2 MIC was 80.13%±1.18%, and the phenyllactic acid at 0.25 MIC and 0.5 MIC had no significant inhibitory effect on the urease activity. It can be analyzed that the MIC was the critical concentration of the phenyllactic acid in inhibiting the urease activity of the H. pylori.

### Example 6 Effects of Ultra Microstructures

### (1) Scanning Electron Microscopy (SEM) Observation

An activated strain H. pylori ZJC03 was gently scraped into 5 mL of a Brucella broth medium with a coating rod, and the OD₆₀₀ was adjusted to 0.5±0.05. A prepared phenyllactic acid solution was added into the H. pylori ZJC03 bacterial solution, and the final concentration of the phenyllactic acid solution was made MIC. An untreated clinical strain H. pylori ZJC03 was used as the control, and static culture was performed for 4 h. Samples were observed after conventional fixing, rinsing, double fixation, dehydration, drying, sample adhesion and coating.

The experimental results are shown in A and B of FIG. 3 which are SEM images of the morphological structures of bacterial cells of the H. pylori not treated with phenyllactic acid. When the H. pylori was not treated by the phenyllactic acid, the bacterial cells of the H. pylori were intact in morphology and clear in color, the morphological structures between the cells were clear and complete, the boundaries between the bacterial cells were clear, and the cells were in a slightly bent rod shape or a short arc shape. As shown in C and D of FIG. 3, when treated with the phenyllactic acid at the MIC, the bacterial cell surfaces of the H. pylori showed wrinkled projections, and the surface became rough. Some bacterial cells were distorted and deformed seriously, some rod-shaped cells became spherical, the internal substances flowed out, most bacterial cells appeared to be disintegrated, and the central area was incomplete or disappeared and even appeared to be flocculent or in the form of a bean curd residue. These results indicated that the phenyllactic acid could disrupt the bacterial envelope system, leading to changes in bacterial cell morphology, cytoplasm efflux and eventual death.

### (2) Transmission Electron Microscopy (TEM) Observation

The preparation of the H. pylori and the loading of the phenyllactic acid were performed as the SEM method described above.

Then samples were observed in a transmission electron microscope Hitachi H-7650 after conventional agar embedding, rinsing, fixation, washing, dehydration, infiltration, re-embedding, sectioning and staining.

A and B of FIG. 4 show the internal structures of cells when the H. pylori is not subjected to phenyllactic acid treatment. The bacterial cell walls and cell membranes of the H. pylori were complete in structure, the electron cloud density was uniform, the nuclear zone was well arranged, most of the bacterial cells were in a bent spiral rod shape, and a small part of the bacterial cells were in a spherical shape. As shown in C and D of FIG. 4, when the H. pylori was treated with the phenyllactic acid at the MIC, the cell morphology changed significantly, most of the H. pylori morphology was spherical, rod-like or U-shaped, the cell surfaces were uneven, and the cell walls were separated from the cell membranes. It can be seen that the cell walls and the cell membranes became thinner and had depressions at many parts, the cytoplasm was unevenly distributed or concentrated and aggregated into clumps, the cytoplasmic electron density was reduced, the nucleoid zones in the center of the cells were seriously deleted, the surrounding contents flowed out, and some cells were broken and cracked and could not be shaped. The results in SEM and TEM in FIGS. 3 and 4 showed that the phenyllactic acid can affect the cell morphology and structure of the H. pylori and cause certain degrees of damage to the surfaces and interiors of the cell membranes, so as to inhibit the H. pylori.

### (3) Transmission Electron Microscope Negative Staining

The H. pylori was activated, a few drops of sterile phenyllactic acid liquid with a concentration of MIC were dripped on a clean glass slide, the H. pylori on Columbia blood agar was slightly scraped with toothpicks and dipped into the phenyllactic acid liquid of the glass slide, such that the H. pylori was dissociated in the phenyllactic acid liquid for 5-10 min and then carefully dripped on a copper net with a Formal film; and the copper net was allowed to stand still, residual liquid was sucked with filter paper, and then the copper net was allowed to stand still for another 2 min. 0.1-0.2 mL of a dye solution was sucked and dripped on the copper net with the bacterial cells, the copper net was allowed to stand still for about 2 min, and the excess dye solution was sucked by filter paper. After the copper net was naturally dried for 5 min, the negative-stain results of the H. pylori before and after the treatment with the phenyllactic acid were observed with a Hitachi transmission electron microscope H-7650.

H. pylori samples were prepared by a pendant-drop negative-stain technique. The morphology observed under the visual field of the transmission electron microscope is shown as A and B of FIG. 5. The H. pylori samples prepared by the pendant-drop negative-stain technique were clear under the visual field of the transmission electron microscope, and the edges of the bacterial cells were clear and visible, without agglomeration and aggregation phenomena. It was found by observation that the H. pylori was in a single-pole shape, had 4-7 flagella with sheaths and blunt ends, could move, had a length of 2.5-4.0 μm and a width of 0.5-1.0 μm, and was in a spiral bent rod shape on the whole. However, the H. pylori treated with the phenyllactic acid at the MIC concentration is shown in C and D of FIG. 5. The flagella of the bacterial cells fell off, the bacterial cells curled up due to the self-protection mechanism, the bacterial cells were ruptured, and the contents flowed out from both sides of the bacterial cells. Vacuoles formed due to the ruptured bacterial cells could be clearly seen in the images, and some bacteria were broken. The results showed that the bacteriostatic effect of the phenyllactic acid on the H. pylori was very likely to cause the falling off of the flagella of the bacterial cells and destroy the cell walls and membranes, enabling the contents to outflow and leading to the death of the bacterial cells.

### Example 7 Grouping of Animal Experiment

After one week of acclimation, mice were randomly divided into 4 groups: ZC, ZH, ZP and ZA. The mice in the groups were treated as follows:

ZC group (15 mice): every mouse was gavaged once every other day with 400 μL of a modified Brucella broth medium for 2 weeks, followed by daily gavage of 400 μL of ultrapure water every day for 4 weeks.

ZH group (15 mice): every mouse was gavaged once every other day with 400 μL of an H. pylori ZJC03 bacterial suspension for 2 weeks, followed by daily gavage of 400 μL of ultrapure water every day for 4 weeks.

ZP group (45 mice): every mouse was gavaged once every other day with 400 μL of the H. pylori ZJC03 bacterial suspension for 2 weeks, followed by daily gavage of 400 μL of phenyllactic acid (at the concentrations of 0.16 mg/mL, 0.32 mg/mL and 0.8 mg/mL respectively, and 15 mice for each concentration) every day for 4 weeks.

ZA group (15 mice): every mouse was gavaged once every other day with 400 μL of the H. pylori ZJC03 bacterial suspension for 2 weeks, followed by 10 days of gavage of combined antibiotics (at the dose of 400 μL per mouse per day), and after 10 days, each mouse continued to be gavaged with 400 μL of ultrapure water for 18 days.

The bacterial content of the H. pylori ZJC03 suspension was 10⁸ CFU/mL.

After the molding, all the mice in each group were sacrificed by cervical dislocation. Gastric mucosa samples were washed with PBS and collected aseptically. The stomach contents were frozen in liquid nitrogen and subsequently stored at −80° C. for testing in Examples 8-10.

The treatment time points of the treatment groups in the above animal experiment are shown in FIG. 6.

### Example 8 Histopathological Observation of Gastric Tissues

The gastric mucosa was fixed in a 4% paraformaldehyde (PFA) buffer solution for 24 h at room temperature. After the treatment, the stomach tissue was embedded with an EG 1160 paraffin machine. The paraffin block was placed in an RM 2235 rotary microtome for sectioning (5 μm), and the sections were stained with hematoxylin and eosin (H&E). The stained sections were observed under a light microscope and photographed (NIKON Eclipse Ci, Japan); and the imaging system was: NIKON digital sight DS-FI2, and the photographic magnification was: 400× and 200×.

Histopathological staining observation is an effective means for diagnosing gastritis, and the staining results of the gastric mucosa of mice in different groups are shown in FIG. 7. From the staining results of the ZC group (A and B of FIG. 7), it can be seen that the mucosal layer of the stomach tissue had clear structure, complete epithelium and closely arranged glands in the mucosal layer, and no other obvious lesions were observed. In the ZH group (C and D of FIG. 7), a small number of necrotic and exfoliated epithelial cells can be observed, gastric gland cell necrosis and karyopyknosis were mostly observed, and a small amount of lymphocyte infiltration was mostly observed; mild edema of the submucosa was rare and a small amount of lymphocyte punctate infiltration was accompanied; and extensive gastric gland dilatation was mostly observed and a small amount of bleeding of the submucosa was occasionally observed. In the ZP group (E and F of FIG. 7) where the mice infected with H. pylori were gavaged with 0.8 mg/mL of phenyllactic acid for treatment, it can be seen from the staining results that the tissue structure was substantially normal, a small number of exfoliated epithelial cells and mild edema accompanied by a small amount of lymphocyte infiltration can be seen in the submucosa, and no other obvious lesions were observed. In the ZA group (G and H of FIG. 7) where the mice infected with the H. pylori were treated with mixed antibiotics, a small number of exfoliated epithelial cells can be observed in the submucosa, and no other obvious lesions were observed.

By comparing the ZP group with the ZA group, it was found that when the concentration of the phenyllactic acid reached 0.8 mg/mL, inflammatory cells basically disappeared, only mild edema accompanied by a small amount of lymphocyte infiltration and few epithelial cell exfoliating phenomena appeared, and the treatment effect of the phenyllactic acid was close to that of the mixed antibiotics.

### Example 9 Real-Time Fluorescence Quantitative PCR (RT-qPCR)

The quantitative analysis of IFN-γ, IL-6, IL-1β and IL-10 in the stomach tissue was performed by RT-qPCR. Total RNA (100-500 ng/μL) was isolated from mouse stomach tissue samples according to the instructions of an RNA extraction kit, followed by reverse transcription of RNA into cDNA using the kit. The levels of each gene were normalized using GAPDH as an internal control. The RT-qPCR assay was performed according to the following thermal cycle program: pre-denaturation at 95° C. for 10 min, 40 cycles of 95° C. for 15 s and 60° C. for 30 s, and the melting curve was from 60° C. to 95° C. with a temperature increase of 0.3° C. every 15 s and the fluorescence signal was collected once, and a 2^{-ΔΔCt} method was used for calculation.

The changes of gastric mucosa inflammatory factors of the mice in the different treatment groups are shown in FIG. 8. Proinflammatory factors IL-1β (A), IFN-γ (B) and IL-6 (C) were all significantly increased, and the anti-inflammatory factor IL-10 (D) was reduced in the ZH group, compared with those in the ZC control group. The results showed that the gavage of the H. pylori could significantly increase the proinflammatory factors and reduce the anti-inflammatory factor. In the ZP group where the mice infected with the H. pylori were gavaged with phenyllactic acid for treatment, IFN-γ, IL-6 and IL-1β were all significantly decreased, and the relative expression level of IL-10 was also increased without a statistical difference (P>0.05), compared with those in the ZH modeling group. These results indicated that the gavage with the phenyllactic acid had a certain therapeutic effect on inflammatory reactions caused by H. pylori infection.

By comparing the ZP group with the ZA group, it was found that the treatments with the phenyllactic acid and the mixed antibiotics can both significantly reduce the gene expression levels of the proinflammatory factors IFN-γ, IL-6 and IL-1β, and the treatment effect of the phenyllactic acid was not significantly different from that of the mixed antibiotics.

### Example 10 Microbiota Analysis Based on 16S rRNA Gene Sequencing Technology

DNA samples were extracted from the gastric mucosa by a CTAB method, and the quality of DNA extraction was detected by agarose gel electrophoresis, and at the same time, the DNA was quantified by an ultraviolet spectrophotometer. The hypervariable regions V3-V4 of 16S rRNA of bacteria were amplified by using universal primers (341F and 805R). Paired-end sequencing of 2×250 bp was performed using a NovaSeq 6000 sequencer, and DNA extraction and sequencing were completed in the LC-Bio Technologies Co. Ltd.

(1) Structure of Gastric Microbiota after Relieving H. pylori Infection by Phenyllactic Acid

16S rRNA gene sequencing was performed by an LC-Bio sequencing platform. The results are shown in A of FIG. 9, where among normal mouse gastric microecological flora, the most abundant phyla were mainly Firmicutes (48.20%), Bacteroides (27.79%), Proteobacteria (13.59%) and Cyanobacteria (4.64%). Meanwhile, among the H. pylori-positive group, Firmicutes (47.75%), Proteobacteria (35.49%), Bacteroides (13.85%) and Cyanobacteria (0.34%) were dominant. Compared with that in the ZH group, the proportion of Proteobacteria was significantly decreased in the treatment groups ZP and ZA, but the ratio of Bacteroides to Firmicutes was significantly decreased in the ZA group, and the ratio of Bacteroides to Firmicutes was increased in the ZP group, which was similarly to that in the normal group. At a genus level, as shown in B of FIG. 9, the microbiota of the ZC group and the ZP group were mainly dominated by Lactobacillus with the relative abundances of 25.63% and 22.39%, respectively, Helicobacter (20.55%) was mainly in the ZH group, and Helicobacter was significantly decreased in the ZA group, but the content of beneficial Lactobacillus in the stomach tissue was also significantly decreased. The above results showed that after H. pylori infection and treatment with phenyllactic acid, the relative abundance of harmful flora was significantly decreased, and the content of Lactobacillus was improved.

By comparing the ZP group with the ZA group, it was found that the phenyllactic acid and the mixed antibiotics can both significantly inhibit Helicobacter in the stomach, but the treatment with the phenyllactic acid can significantly promote the growth of intestinal Lactobacillus at the same time, while the mixed antibiotics had no such effect.

### (2) Alpha and Beta Diversity Analyses of Flora in Gastric Mucosa of Mice in Treatment Groups

Alpha diversity indexes are used to characterize microflora diversity within samples. As shown in Table 3, the flora abundance and diversity of the gastric mucosa of the mice in the modeling group and the treatment groups were changed to a certain extent compared with those in the control group. Therefore, it can be seen that the treatment with the phenyllactic acid can improve the decreased abundance of microbial species in the gastric mucosa caused by the H. pylori infection. In average Simpson index and Shannon index analyses, the indexes in the ZC group were higher than those in the ZH group and other treatment groups, followed by the phenyllactic acid high-concentration treatment group, which indicated that the treatment with the phenyllactic acid can improve the decrease of the diversity of the microbial flora in the gastric mucosa caused by the H. pylori infection.

TABLE 3
Comparison of a diversity parameters between
treatment groups and control group

| | Number | Observed | | | |
|---|---|---|---|---|---|
| Group | of mice | species | Chao1 | Simpson | Shannon |
| ZH group | 15 | 341.2 | 376.14 | 0.863966948 | 5.325325879 |
| ZC group | 15 | 556.4 | 575.85 | 0.933441475 | 6.410253254 |
| ZP group | 15 | 453.6 | 464.56 | 0.928733531 | 6.139339081 |
| ZA group | 15 | 406.4 | 418.50 | 0.936370341 | 5.968263861 |

Note: ZA is an antibiotic treatment group, ZC is a control group, ZH is a modeling group, and ZP is a 0.8 mg/mL phenyllactic acid treatment group

Beta diversity is often used to compare diversities between different ecosystems. The difference between samples of each treatment group is shown generally by using a Weighted UniFrac PcoA method. As shown in FIG. 10, the contribution rate of PC1 was 34.99%, the contribution rate of PC2 was 23.79%, and the sum of both contribution rates was greater than 50%, which indicated that the difference among the samples was not caused by external interference, but caused by the samples themselves, and the detection result was significant. By comparing the distances between various sample points in the graph, the difference between individuals or groups is observed, and the samples closer to each other in the coordinate graph have a greater similarity and a smaller difference. The samples of the ZC, ZP and ZA groups were substantially similar except for individual samples with deviations, and were mainly distributed in the area close to the center, while the samples of the ZH group, namely the H. pylori infection group, were far apart. Therefore, it can be seen that the ZC group and the phenyllactic acid high-concentration treatment group had relatively large differences from the ZH group, which also indicated sidewise that the changes in the gastric microbial flora caused by the H. pylori can be improved by treating the mice by gavage with phenyllactic acid to some extent.

By comparing the ZP group with the ZA group, it was found that both the treatments with the phenyllactic acid and the mixed antibiotics had no significant effect on the diversity of the gastric flora in the mice.

### (3) Significance Difference Analysis of Species in Gastric Mucosa of Mice in Treatment Groups

After 4 weeks of the phenyllactic acid treatment, significant differences in the flora relative abundance and the bacterial genus among the four groups, i.e. the ZC, ZH, ZP and ZA groups, were linearly discriminated by using an LEfSe method (LDA>3). As shown in FIG. 11 (A and B), in the ZH group, Proteobacteria was the dominant phylum, Helicobacteraceae, Vibrionaceae, Pasteurellaceae, Acetobacteraceae and Paenibacillaceae were significantly increased dominant families, and at the “genus” level, Helicobacter, Vibrio, Rodentibacter, Cupriavidus, Paenibacillaceae and Coriobacteriaceae UCG 002 were significantly increased, where the average relative abundance of Helicobacter pylori in the stomach of the infected mice was increased by 17.29% compared with the normal group; and in the gastric microbial flora of the ZP group, the flora with significant increases were Prevotella, Lactobacillus, Faecalibaculum, Olsenella, Aerococcus etc. at the “genus” level, and the relative abundance of the Helicobacter pylori was decreased by 9.46% compared with that in the ZH group, and in addition, Lactobacillus at the “order” level was most significantly increased in the ZP group and most Lactobacillus species are known to be isolated from the gastrointestinal tract of humans and animals. It can be seen from the above that the therapeutic effect of the phenyllactic acid improved the abundances of probiotics such as Lactobacillus, Prevotella, Faecalibaculum and Olsenella in the gastric mucosa of the mice and reduced the abundances of pathogenic bacteria such as Vibrio and Helicobacter.

By comparing the ZP group with the ZA group, it was found that the treatment with the phenyllactic acid was more beneficial to the growth of the beneficial gastric flora Lactobacillus, Prevotella, Faecalibaculum, Olsenella, etc.

In conclusion, in the present invention, on the basis of the in vitro bacteriostatic experiments, SPF-grade mice were selected to establish an H. pylori gastritis infection model, and the effect of the phenyllactic acid on the gastric microecology of the H. pylori infected mouse model was explored by using a method for treating this gastritis model by gavage with the phenyllactic acid. The following results were obtained:

1. it was proved that the phenyllactic acid had stronger in vitro bacteriostatic activity on the H. pylori from five aspects of the inhibition zone, MIC, growth curve, ultra microstructure and urease activity. The experimental results showed that the MIC was 2.5 mg/mL, the phenyllactic acid at the MIC completely inhibited the growth of the H. pylori. The inhibitory effect was gradually weakened with the decrease of the phenyllactic acid concentration, and the inhibition rate of the phenyllactic acid at the MIC on the urease of the H. pylori was up to 43.01%+2.11%. The bacterial cell morphology of the H. pylori ZJC03 treated by the phenyllactic acid at the MIC was observed through SEM and TEM, and it was found that the bacterial cells deformed, the cell wall membranes were severely damaged, and even the nucleoid zones in the center of the cells were deleted.

2. C57BL/6J mice were selected to establish an H. pylori infected mouse model. Through the H&E staining, RT-qPCR and other methods, inflammatory cell infiltration and inflammatory factor change levels in the gastric mucosa were detected to explore whether the phenyllactic acid had an alleviating effect on the gastritis symptoms of the mice. The results showed that the treatment with the phenyllactic acid can reduce the infiltration of inflammatory cells in the gastric mucosa of the mice and significantly down-regulate the expression level of proinflammatory cytokines. It was indicated that the phenyllactic acid can alleviate the gastritis symptoms caused by the H. pylori infection through regulating host immune response and reducing oxidative damage.

3. the phenyllactic acid had a regulatory effect on the gastric flora disorder induced by the H. pylori. The therapeutic effect of the phenyllactic acid significantly improved the abundances of beneficial bacteria such as Lactobacillus, Prevotella and Faecalibaculum in the gastric mucosa of the mice and reduced the abundances of pathogenic bacteria such as Vibrio and Helicobacter. Compared with the antibiotic treatment group, the increase in the beneficial flora in the gastric mucosa of the mice in the phenyllactic acid treatment group was more significant. The phenyllactic acid can improve the gastric microecological flora disorder in the organism caused by the H. pylori infection and regulate the gastric microecological balance.

In the process of invention, the following comparative experiment was further performed in the present invention: lactic acid (with a broad-spectrum bacteriostatic property) was used to replace the phenyllactic acid, and the detection was performed according to the method described in Example 2. The obtained results were that 10 mg/ml of lactic acid could not inhibit the growth of the H. pylori ZJC03 (insensitive to metronidazole), and there was no visible inhibition zone; but the results obtained with the present invention were that 10 mg/mL of the phenyllactic acid can significantly inhibit the growth of the H. pylori, and the diameter of the inhibition zone was 9.98±0.97 mm.

Finally, it should be noted that the examples listed above are merely several specific examples of the present invention. Obviously, the present invention is not limited to the above examples and can have many variations. All the variations that can be directly derived or conceived by a person of ordinary skill in the art from the disclosed content of the present invention should be regarded as falling into the protection scope of the present invention.

What is claimed is:

1. A method for inhibiting Helicobacter pylori infection, wherein
when the method is used for inhibiting the Helicobacter pylori infection in vivo, the method comprises orally administrating phenyllactic acid at a dose of 2.5-3.5 mg/person per day, thereby promoting growth of intestinal Lactobacillus; or
when the method is used for inhibiting the Helicobacter pylori infection in vitro, the method comprises diluting phenyllactic acid to 2.5-40 mg/mL with ultrapure water, thereby achieving a bacteriostatic effect on Helicobacter pylori.

2. The method for inhibiting Helicobacter pylori infection according to claim 1, wherein the phenyllactic acid is capable of inhibiting growth of Helicobacter pylori insensitive to metronidazole.

3. The method for inhibiting Helicobacter pylori infection according to claim 1, wherein the phenyllactic acid has at least one of the following properties:
the phenyllactic acid is capable of inhibiting the growth of the Helicobacter pylori;
the phenyllactic acid relieves gastric mucosal inflammation caused by the Helicobacter pylori infection;
the phenyllactic acid inhibits urease activity of the Helicobacter pylori; and
the phenyllactic acid is destructive to a bacterial cell of the Helicobacter pylori.

4. The method for inhibiting Helicobacter pylori infection according to claim 2, wherein the phenyllactic acid has at least one of the following properties:
the phenyllactic acid is capable of inhibiting the growth of the Helicobacter pylori;
the phenyllactic acid relieves gastric mucosal inflammation caused by the Helicobacter pylori infection;
the phenyllactic acid inhibits urease activity of the Helicobacter pylori; and
the phenyllactic acid is destructive to a bacterial cell of the Helicobacter pylori.
